(19) United States
(12) Patent Application Publication
(10) Pub. No.: US 2006/0224040 A1
(43) Pub. Date: Oct. 5, 2006

(54) In vivo imaging device and method of manufacture thereof

(75) Inventors: Semion Khait, Tiberias (IL); Zvika Gilad, Haifa (IL)

Correspondence Address: PEARL COHEN ZEDEK, LLP, 1500 BROADWAY 12TH FLOOR, NEW YORK, NY, 10036, US

(73) Assignee: GIVEN IMAGING LTD.

(21) Appl. No.: 11/094,288

(22) Filed: Mar. 31, 2005

(51) Int. Cl. A61B 1/05

(52) U.S. Cl. 600/102

(57) ABSTRACT

An in vivo imaging device including one or more components for example an imager, a transmitter and a circuit board having rigid sections and flexible sections According to some embodiments, the in vivo imaging device components may be electrically joined and/or stacked together using three-dimensional (3D) chip scale packaging solutions.

### FIELD OF THE INVENTION

The present invention relates to an in vivo imaging device and system, such as, for example, for imaging the digestive tract or other body lumens.

### BACKGROUND OF THE INVENTION

Known devices may be helpful in providing in-vivo imaging Autonomous in-vivo imaging devices, such as swallowable or ingestible capsules or other devices may move through a body lumen, imaging as they move along, Some of these devices use a wireless connection to transmit image data.

In some in vivo devices, such as ingestible imaging capsules, the components within the capsule, such as an imager(s), may be arranged on a support and/or on a board or on several boards, for example on a printed circuit board (PCB). In some cases the boards are aligned along an axis of the capsule and are electrically connected by a plurality of wires.

Several factors have so far limited the extent to which the size, weight and power consumption of an imaging device can be reduced. A first factor may be the size of the components and the boards and/or the support e.g. the PCB located in the device. Another factor limiting the size, weight and energy reduction or space usage in imaging devices may be the number of integrated components. A third factor may be the average spacing between the components.

### SUMMARY OF THE INVENTION

The present invention provides, according to some embodiments, an in vivo imaging device comprising a support, such as a circuit board having one or more rigid sections or portions, and one or more flexible sections or portions. In some embodiments, the rigid sections and flexible sections may alternate.

According to some embodiments of the present invention, the in vivo imaging device may include an image sensor. The device may further include an illumination system and/or a transmitter an antenna for transmitting (and/or for receiving) image data to a receiving system and a processor.

According to some embodiments of the present invention, some components in the device, for example, the imager and/or the transmitter and/or the processor may be vertically mounted and/or stacked on the circuit board, and may be further interconnected to each other.

According to some embodiments of the present invention, the support, for example the circuit board may be manufactured or pre-provided to include one or more three-dimensional (3D) electrical packages for vertically packaging the components of the in-vivo device and so as to possibly reduce the amount of space taken up by the components. According to some embodiments of the present invention, 3D chip scale packaging solutions may help to meet size and performance requirements of the in-vivo imaging device by providing the following benefits, for example: reduction of size and weight in the package—vertical stacking may reduce the number of chip-to-board (e.g. component-to-circuit board) interconnections and the area required for chips and/or components; reduction in power consumption—the level of power required depends in part on the number of interconnects; increase in performance and reliability—reducing the number of module-to-board solder connections by using 3D components scale packaging may decrease board failures

### BRIEF DESCRIPTION OF THE DRAWINGS

The invention is herein described, by way of example only, with reference to the accompanying drawings, in which like components are designated by like reference numerals, wherein:

FIG. 1 shows a schematic diagram of an in vivo imaging device according to one embodiment of the present invention;

FIGS. 2A and 2B schematically illustrate a top side view and a bottom side view, respectively, of a circuit board in accordance with the present invention;

FIG. 3 shows a schematic diagram of an in vivo imaging device according to another embodiment of the present invention;

FIGS. 4A and 4B schematically illustrate a top side view and a bottom side view, respectively, of a circuit board in accordance with another embodiment of the present invention;

FIGS. 5A-5C illustrate a cross-sectional view of a 3D package in accordance with another embodiment of the present invention,

FIG. 6 is a schematic flow-chart of a method of manufacturing an in vivo imaging device in accordance with some embodiments of the invention; and

FIG, 7 is a schematic flow-chart of a method of manufacturing three-dimensional electrical device packages in accordance with some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

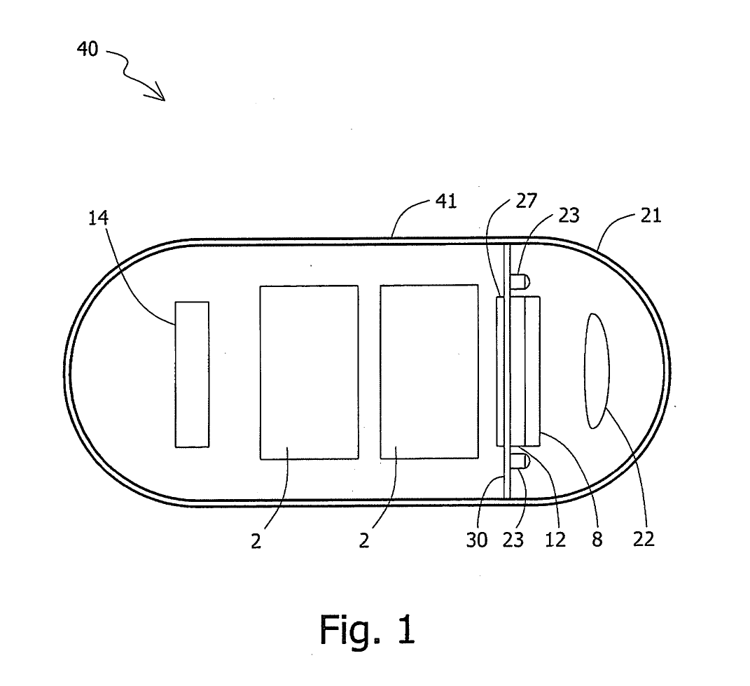

Reference is now made to FIG. 1, which schematically illustrates an in vivo imaging device according to an embodiment of the present invention. According to one embodiment of the present invention, the device 40 may include an optical window 21 and an imaging system for obtaining images from inside a body lumen, such as the GI tract. The in-vivo device 40 may include a container or housing 41 Within the housing 41, may be, for example, the imaging system which may include one or more illumination sources 23, such as a white LED (Light Emitting Diode) and/or an OLED (Organic LED), an image sensor 8, such as a CMOS imaging camera and an optical system 22 which focuses light onto the CMOS image sensor 8. The illumination source 23 illuminates the inner portions of the body lumen through optical window 21. Device 40 may further include a transmitter 12 and an antenna 27 for transmitting image signals from the CMOS image sensor 8, and a power source 2, such as a silver oxide battery, that provides power to the electrical elements of the device 40. According to some embodiments of the present invention, device 40 may include a processing unit separate from transmitter 12 that may, for example, contain or process instructions. Optionally, according to one embodiment of the present invention, transmitter 12 may include a processing unit or processor or controller, for example, to process signals and/or data generated by imager 8. In another embodiment, the processing unit may be implemented using a separate component within device 40, e.g., controller or processor 14, or may be implemented as an integral part of imager 8, transmitter 12, or another component, or may not be needed. The optional processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit, In one embodiment, for example, the processing unit or controller may be embedded in or integrated with transmitter 12, and may be implemented, for example, using an ASIC.

According to some embodiments of the present invention, device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power or transmit information. In one embodiment, all of the components may be sealed within the device body (the body or shell may include more than one piece); for example, an imager, illumination units, power units, and transmitting and control units, may all be sealed within the device body.

The system and method of the present invention may be used with or in an imaging system such as that described in U.S. patent application, Ser. No. 09/800,470, entitled A DEVICE AND SYSTEM FOR IN-VIVO IMAGING, filed on Mar. 8, 2001. A further example of an imaging system with which the system and method of the present invention may be used is described in U.S. Pat. No. 5,604,531 to Iddan et al., entitled IN-VIVO VIDEO CAMARA SYSTEM, filed on Jan. 17, 1995. Both these publications are assigned to the common assignee of the present application and are hereby incorporated by reference. Alternatively, the system of the present invention may be utilized in any suitable imaging device providing images of a body lumen or cavity For example, a circuit board according to an embodiment of the invention may be utilized in probes used for in vivo imaging, such as endoscopes

According to one embodiment of the present invention, the various components of the device 40 may be disposed on a support, for example a circuit board 30. According to some embodiments of the present invention, the in vivo imaging device components may be electrically joined and/or stacked together using three-dimensional (3D) chip scale packaging solutions. 3D chip scale packaging refers to a vertical (Z-axis) stacking of multiple die within a package, or multiple packages, using specialized substrates and/or interconnects. According to some embodiments of the present invention, the in vivo imaging device components, for example the imager 8 and/or the transmitter 12 may be interconnected using different vertical interconnection methods and techniques used in 3D packaging, for example a Stacked tape carrier, a Solder edge conductor bonding, Folded Flex Circuits, Thin Film Conductors on Face-of-a-Cube, wire bonded stacked chips.

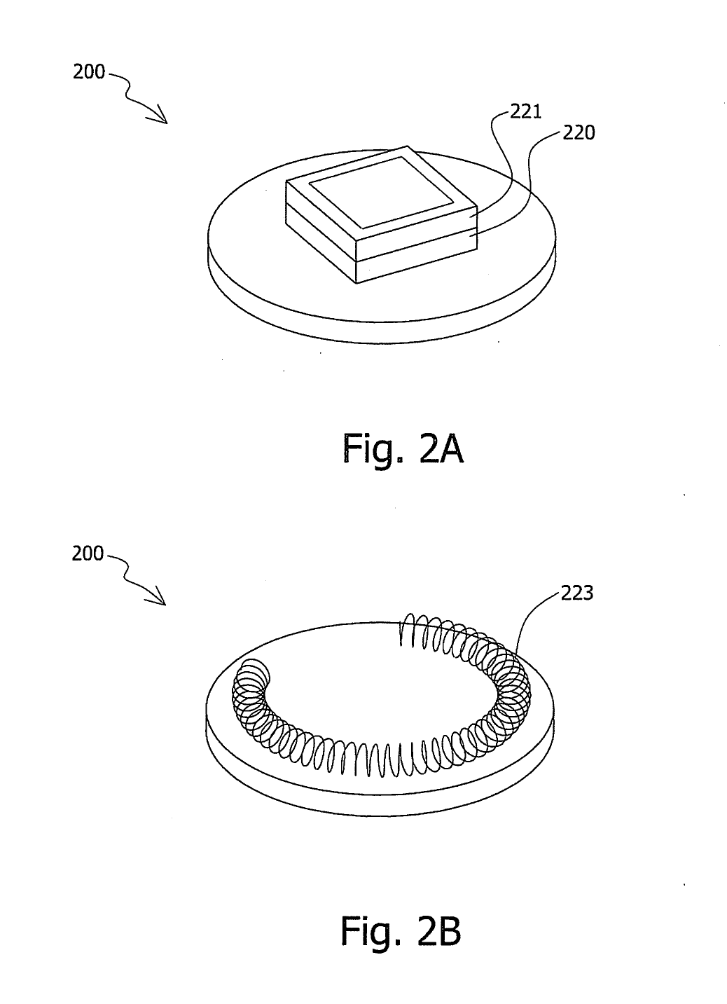

FIGS. 2A and 2B schematically illustrate a top side view and a bottom side view, respectively, of a circuit board or other suitable holder 200 in accordance with some embodiments of the invention. In some embodiments, circuit board 200 may be an example of circuit board 30 of FIG. 1. In some embodiments, circuit board 200 may be used in conjunction with device 40 of FIG. 1, or with other suitable devices and systems for in vivo sensing or in vivo imaging.

According to some embodiments of the present invention, the circuit board 200 may include an imager 221, a transmitter such as an ASIC 220 and an antenna 223.

According to some embodiments of the present invention, the in-vivo sensing device components such as the imager 221 and the ASIC 220 may be connected to one another by using one or more Vertical Interconnections techniques. Vertical Interconnections refer to the interconnections needed, for example to route power, ground, and signals to the components within the in-vivo device.

According to some embodiments of the present invention, one or more components of device 40, for example the imager 221 and the ASIC 220 may be attached and/or interconnected for example, to the circuit board 200 using 3D chip scale packaging techniques. For example, according to one embodiment of the present invention, the imager 221 the ASIC 220 and the circuit board may be interconnected to one another by using, for example a bonding layer such as a Solder Bumps layer.

According to the above-described configurations of the circuit board 200 and/or the in-vivo device 40, a circuit board 200 and the in vivo device 40 can be formed smaller than existing devices, with thinner packages and more silicon functions per cm² and more silicon functions per cm³ of in-vivo application space, thereby realizing an in-vivo device which is light, small and with reduced power consumption.

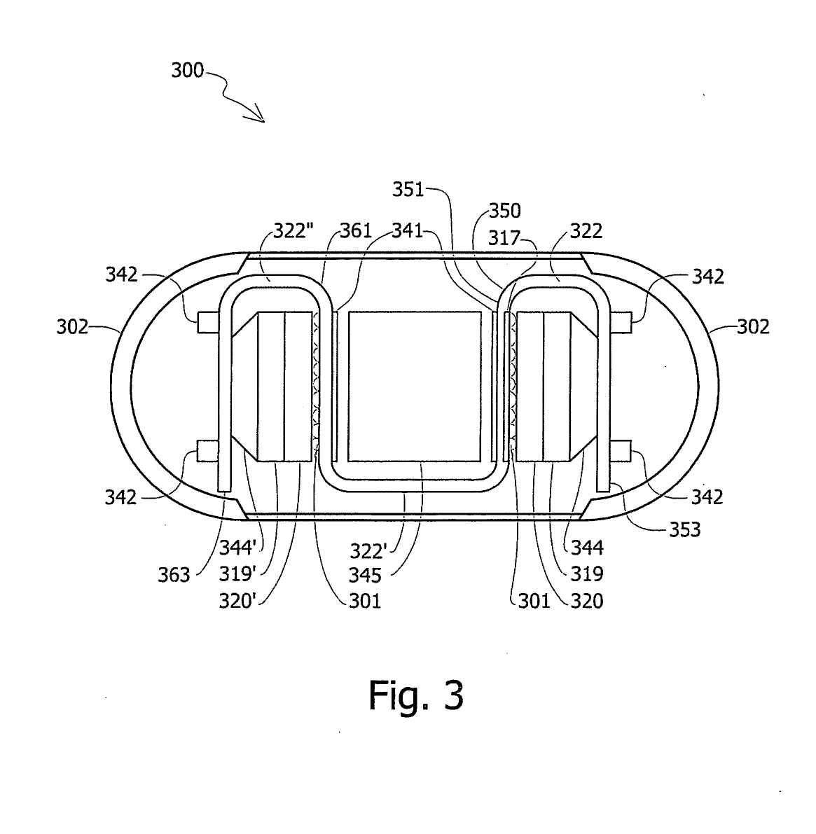

Another embodiment of the invention is schematically illustrated in FIG. 3, in which a longitudinal cross section of device 300 is schematically shown. According to one embodiment of the present invention, device 300 may include two optical domes 302 behind which are situated illumination sources 342, two lens holder 344 and 344′, two imagers 319 and 319′ a transmitter such as an ASIC 320 and a processor 320′. The device 300 may further include a power source 345, which may provide power to the entirety of electrical elements of the device, an antenna 317 for transmitting video signals from the imagers 319 and 319′. According to some embodiments of the present invention, device 300, is capable of simultaneously obtaining images of the body lumen, for example, the GI tract, from two ends of the device. For example, device 300 may be a cylindrical capsule having a front end and a rear end, which is capable of passing the entire GI tract. The system in a cylindrical capsule can image the GI tract in the front and in the rear of the capsule.

According to one embodiment of the present invention, the various components of the device 300 may be disposed on a circuit board 350 including rigid and flexible portions, preferably the components are arranged in a stacked vertical fashion. For example, rigid portion 351 of the circuit board 350 may hold a transmitter 320, an imager 319 and a lens holder 344, while rigid portion 361 may hold a processor 320′, an imager 319′ and a lens holder 344′; the other side of the rigid portions 351 and 361 may include, for example, a contact 341 for battery or power source 345. According to one embodiment of the present invention, rigid portions 353 and 363 of the circuit board 350 may include, for example, an illumination source, such as one or more LEDs 342 or other illumination sources According to some embodiments of the present invention, each rigid portion of the circuit board may be connected to another rigid portion of the circuit board by a flexible connector portion (e.g. 322 322′ and 322″) of the circuit board 350. According to one embodiment of the present invention, each rigid portion of the circuit board may include two rigid sections; sandwiched between the rigid sections is a flexible connector portion of the circuit board for connecting the rigid boards. In alternate embodiments, other arrangements of components may be placed on a circuit board having rigid portions connected by flexible portions.

In alternate embodiments, a circuit board having rigid portions and flexible portions may be used to arrange and hold components in other in vivo sensing devices, such as a swallowable capsule measuring pH, temperature or pressure, or in a swallowable imaging capsule having components other than those described above. Such circuit boards may be similar to embodiments described in U.S. application Ser. No. 10/879,054 entitled IN VIVO DEVICE WITH FLEXIBLE CIRCUIT BOARD AND METHOD FOR ASSEMBLY THEREOF, and U.S. application Ser. No. 60/298,387 entitled IN VIVO SENSING DEVICE WITH A CIRCUIT BOARD HAVING RIGID SECTIONS AND FLEXIBLE SECTIONS, each incorporated by reference herein in their entirety.

According to some embodiments of the present invention, one or more components of device 300, for example the lens holders 344 and 344′, the imagers 319 and 319′ the transmitter 220 and the processor 220′ may be packaged and may be further attached and/or interconnected for example, to the circuit board 350 using 3D chip scale packaging techniques. For example, according to one embodiment of the present invention, the lens holder 344, the imager 319, the transmitter 320 and the circuit board 350 may be interconnected to one another by using, for example a bonding layer such as a Solder Bumps layer 301.

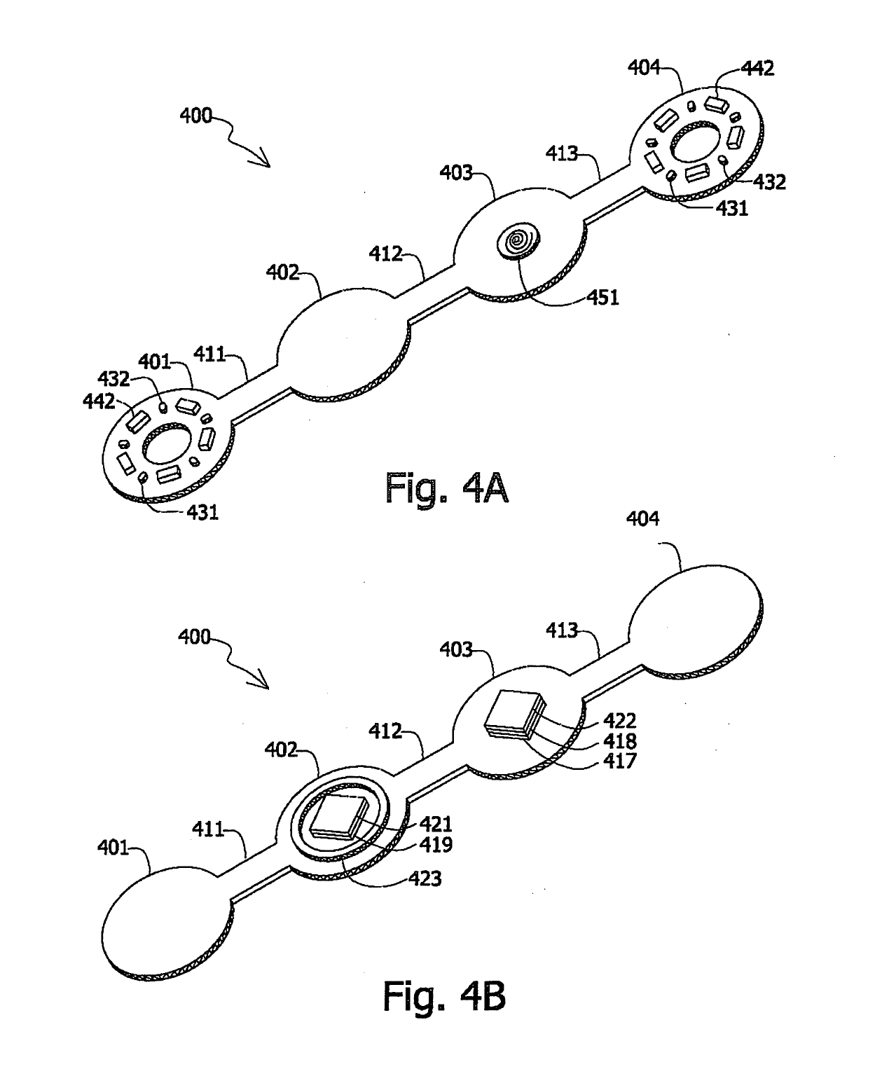

FIGS. 4A and 4B schematically illustrate a top side view and a bottom side view, respectively, of a circuit board 400 in accordance with some embodiments of the present invention. In some embodiments, circuit board 400 may be an example of circuit board 300 of FIG. 3. In some embodiments, circuit board 400 may be used in conjunction with device 300 of FIG. 3, or with other suitable devices and systems for in vivo sensing or in vivo imaging.

According to one embodiment of the present invention circuit board 400 may include, for example, one or more rigid portions and one or more flexible portions. For example, circuit board 400 may include rigid portions 401, 402, 403 and 404, which may be interconnected using flexible portions 411, 412 and 413. Although four rigid portions and three flexible portions are shown, embodiments of the present invention are not limited in this regard, and may include other numbers, orders or combinations of rigid portions and/or flexible portions.

In some embodiments, rigid portion 401 and/or rigid portion 404 may include, for example, one or more illumination units or LEDs 442, and optionally one or more resistors 431 and/or capacitors 432 to regulate or control the power provided to illumination units or LEDs 442. Although two rigid portions 401 and 442 having illumination units or LEDs 442 are shown, embodiments of the invention are not limited in this regard; for example, in one embodiment, circuit board 400 may include rigid portion 401 and may not include rigid portion 404.

In some embodiments, rigid portion 402 may include a first imager 421, a transmitter such as an ASIC 419 and an antenna 423. In some embodiments, rigid portion 403 may include a battery holder 451, e.g., a spring able to hold a battery or other power source in place. According to some embodiments of the present invention, rigid portion 403 may optionally include a second imager 422 and/or a processor 418 and/or a memory 417. Although two imagers 421 and 422 are shown, embodiments of the invention are not limited in this regard, for example, in one embodiment, circuit board 400 may include one imager, or another suitable number of imagers.

According to some embodiments of the present invention, the various components of the device 300, for example the components which are disposed on the circuit board 400 may be electrically interconnected using three-dimensional (3D) chip scale packaging solutions. For example, according to one embodiment of the present invention, the imager 422 and the ASIC 419 may be vertically packaged using a vertical interconnection techniques, for example a Stacked tape carrier or Solder edge conductor bonding. According to one embodiment of the present invention, the Imager 422 and/or the processor 418 and/or the memory 417, may be interconnected to each other, and mounted to the circuit board 400 using 3D stacking techniques, such as a Stacked tape carrier, Solder edge conductor bonding, Folded Flex Circuits, Thin Film Conductors on Face-of-a-Cube wire bonded and stacked chips methods.

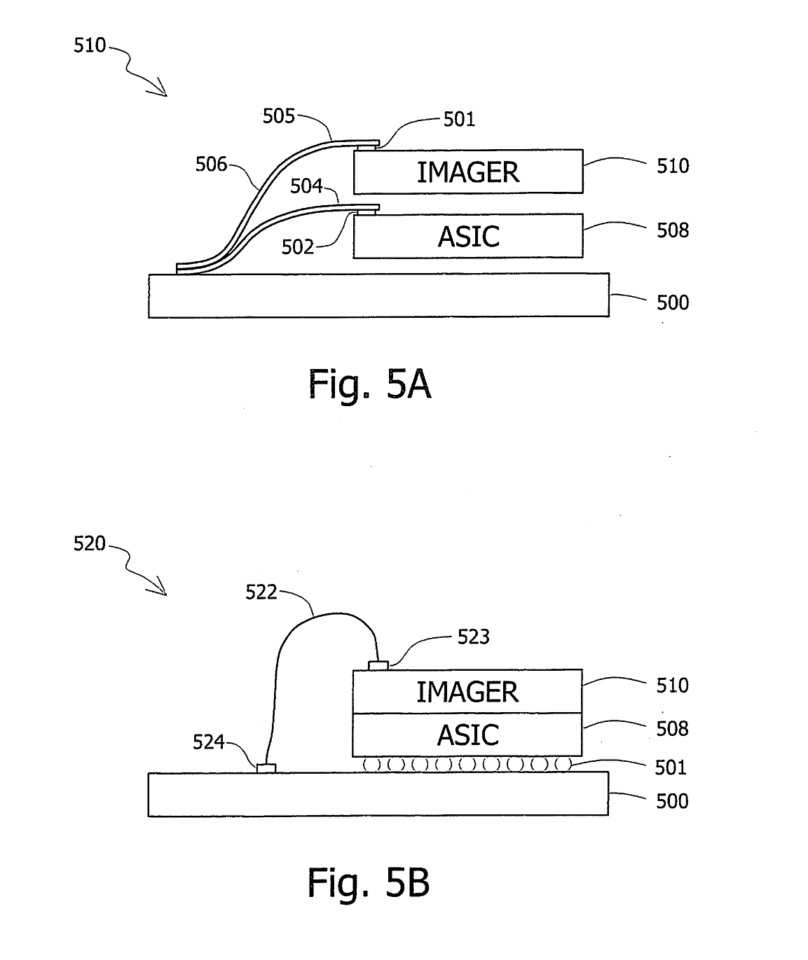

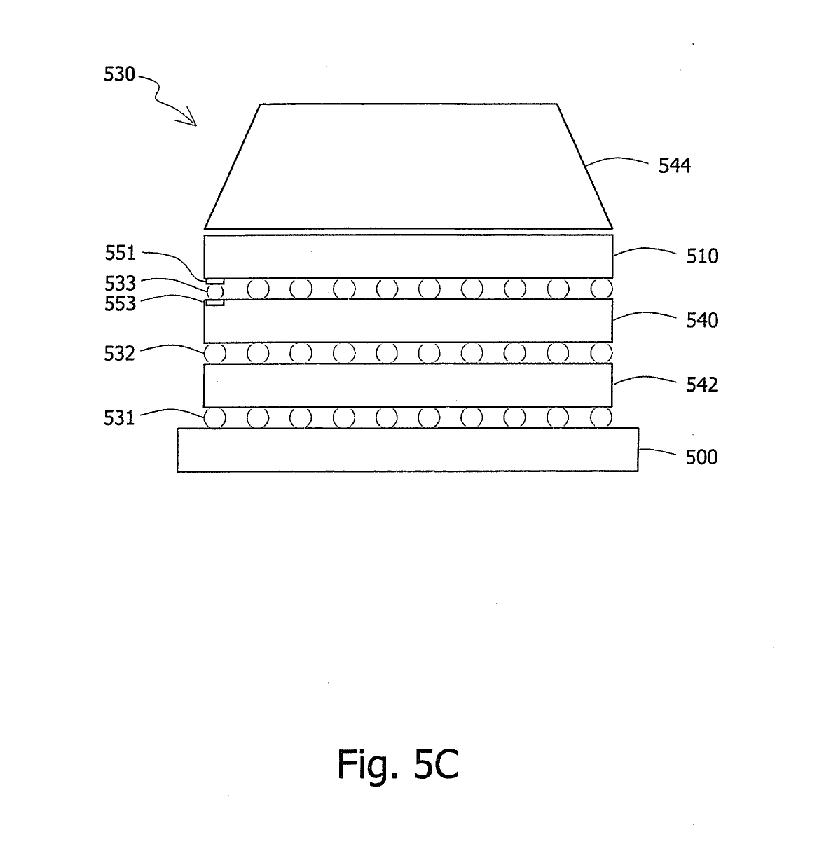

FIG. 5A illustrates a cross-sectional view of a 3D package 510, according to one embodiment of the present invention. According to one embodiment of the present invention, the 3D package 510 may include two or more components and/or chips interconnected to each other using 3D packaging solutions. According to one embodiment of the present invention, the 3D package may include an imager 510 and an ASIC 508. According to some embodiments of the present invention, the components are disposed on a support such as a circuit board, for example in a stacked vertical fashion, and may be interconnected to each other using 3D packaging solutions. According to one embodiments of the present invention the various components of the in-vivo device, for example the imager 510 and the ASIC 508 may be interconnected, for example by a stacked tape carrier 506. A Stacked tape carrier is a method for interconnecting ICs using TAB technology (Tape-Automated Bonding). For example, according to one embodiment the stacked tape carrier 506 may include one or more TAB leads 505 and 504 which may be connected by pads 501 and 502 to imager 510 and ASIC 508.

FIG. 5B illustrates a cross-sectional view of a 3D package 520, according to one embodiment of the present invention. According to one embodiment of the present invention, the 3D package may include an imager 510 and an ASIC 508. According to some embodiments of the present invention, the components are disposed on a circuit board, for example in a stacked vertical fashion, and may be interconnected to each other using 3D packaging solutions. According to one embodiments of the present invention the various components of the in-vivo device, for example the imager 510 and the ASIC 508 may be interconnected, for example by a wire bonding interconnection and/or a solder balls layer and/or a Flip Chip. For example, according to one embodiment, the imager 510 may be interconnected to circuit board 500 using a wire conductor 522. According to one embodiment, the wire may be used to electrically connect the imager to the circuit, for example by one or more pads such as an On-chip pad 523 and a substrate pad 524. According to one embodiment of the present invention, the ASIC 508 may be interconnected to the circuit board 500 through a connection layer, for example a bonding layer 501.

FIG. 5C illustrates a cross-sectional view of a 3D package 530, according to another embodiment of the present invention. According to one embodiment of the present invention the 3D package 530 may include one or more components, for example an optical system such as a lens holder 544, an imager 510 a memory 540 and/or a buffer 542 and a circuit board 500. According to one embodiment of the present invention, the electric devices such as the imager 510 the memory 540 and/or the buffer 542 and a circuit board 500 may be interconnected for example by one or more layers and/or conductive paths such as ACE (Anisotropic Conductive Elastomer) layers 531, 532 and 533. According to one embodiment of the present invention, the layers may be located between the adjacent electrical devices, and between the lowest electrical device and the circuit board.

According to some embodiment of the present invention, the layers such as the ACE layers 531, 532 and 533 may provide electrical interconnection along the vertical electrical bus comprised of electrical contacts and circuits such as contacts 551 and 553 that are on the upper and lower surface of each ACE layer and/or on the adjacent devices 531, 532 and 533. This may provide the necessary and desired inter-layer electrical contact through the stack The ACE layers are both electrically and thermally conductive in the vertical direction due to the embedded conductive metal elements. The vertical bus includes contact zones on the top and bottom surface of each individual electrical device and package, as appropriate, which may be used to provide inter-layer electrical contact. According to one embodiment package 530 may consist of several independent packages, or several devices making up a single package.

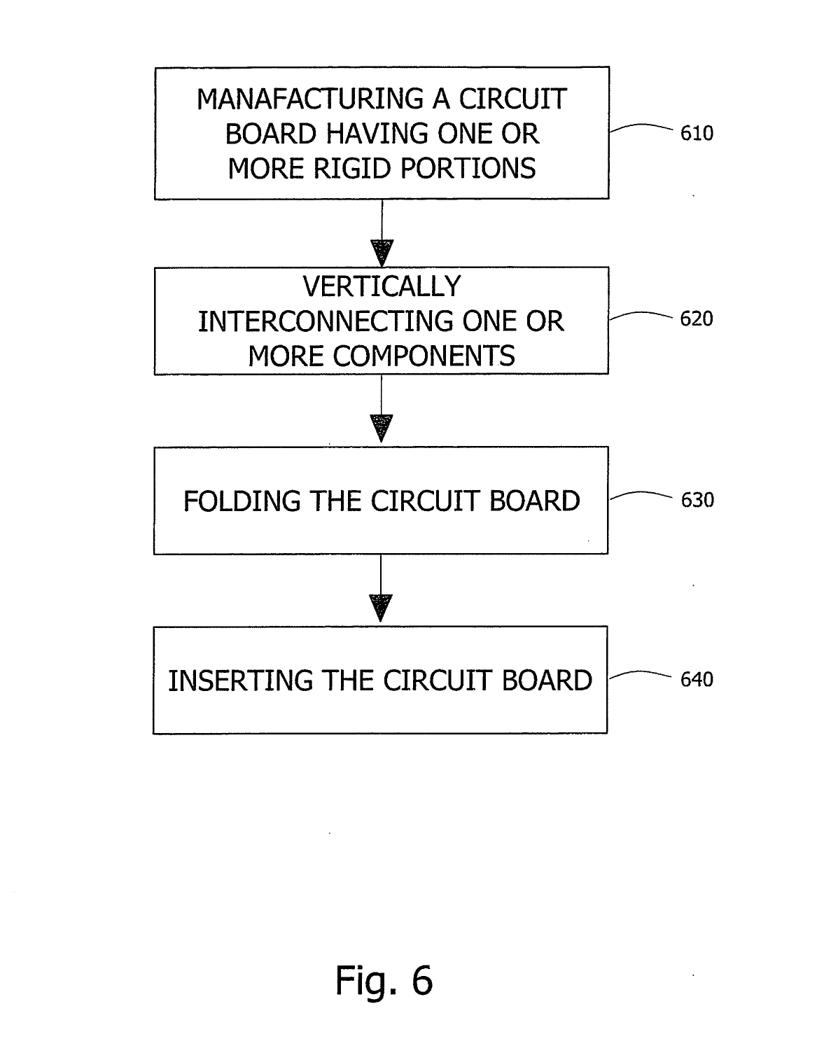

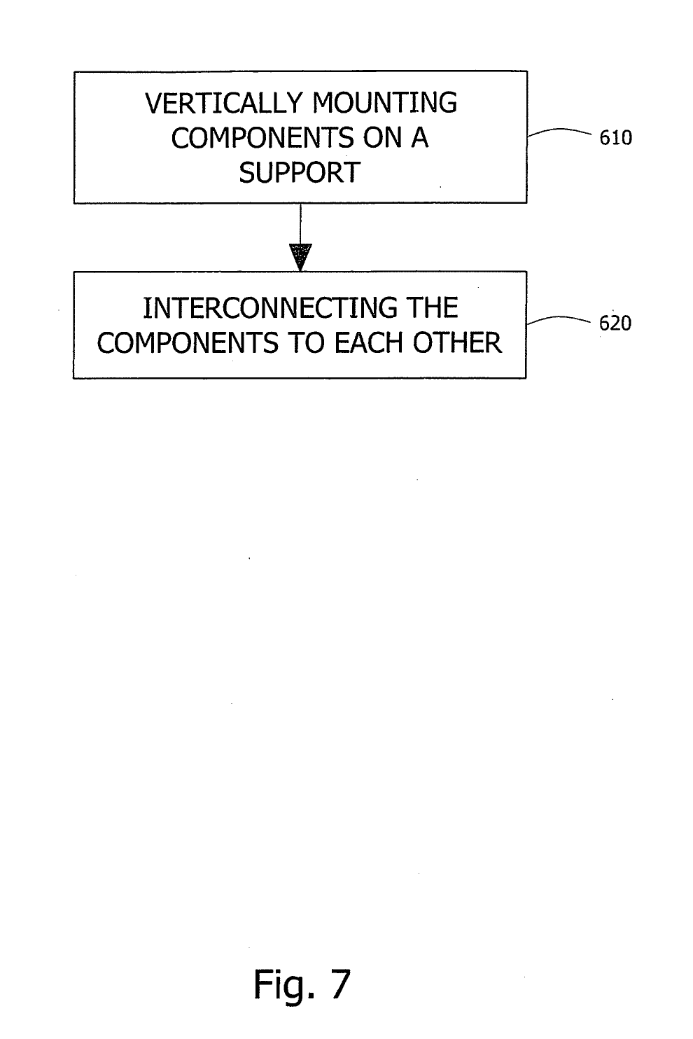

FIG. 6 is a schematic flow-chart of a method of manufacturing an in vivo imaging device in accordance with some embodiments of the invention. As indicated at box 610, the method may include manufacturing or providing a support, for example a circuit board having one or more rigid portions and one or more flexible portions As indicated at box 620, the method may optionally include vertically attaching or interconnecting one or more components, for example to a rigid portion of the circuit board. This may include, for example, attaching a lens holder, an imager, an ASIC, a memory, a buffer or other suitable components. As indicated at box 630, the method may include folding, bending, twisting and/or shaping of the circuit board or a flexible portion of the circuit board, for example, into a pre-defined shape.

As indicated at box 640, optionally, the method may include inserting the folded circuit board into a suitable housing adapted or configured for in vivo imaging, for example, a housing of a swallowable capsule. Other suitable operations or methods may be used in accordance with embodiments of the invention.

FIG. 7 is a schematic flow-chart of a method for the manufacture of a three-dimensional electrical device package in an in-vivo imaging device. As indicated at box 710, the method may include vertically mounting and/or stacking one or more components on a support, for example stacking a transmitter and an imager on a circuit board. As indicated at box 620, the method may optionally include interconnecting said components to each other, for example to a rigid portion of the circuit board. This may include, for example, interconnecting the components using different vertical interconnection methods and techniques used in 3D packaging, for example a Stacked tape carrier, a Solder edge conductor bonding, Folded Flex Circuits, Thin Film Conductors on Face-of-a-Cube, wire bonded stacked chips, and a Folded Flex Circuit.

It will be appreciated by persons skilled in the art that the present invention is not limited to what has been particularly shown and described hereinabove. Rather, the scope of the present invention is defined by the claims which follow.

1. An autonomous in vivo imaging device comprising a housing and a plurality of components packaged vertically upon a single support within the housing.

2. The in vivo imaging device of claim 1, wherein said components are selected from the group consisting of: an imager, a transmitter, a memory, a circuit board and a buffer.

3. The in vivo imaging device of claim 1, wherein said support is a circuit board.

4. The in vivo imaging device of claim 1, wherein said support comprises a plurality of rigid sections and a plurality of flexible sections.

5. The in vivo imaging device of claim 4, wherein said plurality of components are positioned on a rigid section of said support.

6. The in vivo imaging device of claim 1, comprising a lens holder.

7. The in vivo imaging system of claim 1, wherein said in vivo imaging device comprises a swallowable capsule.

8. An autonomous in vivo imaging device comprising a package of vertically stacked multiple die, the die being electrically interconnected.

9. The device according to claim 8 wherein the die include components of the device.

10. The device according to claim 8, wherein the components are selected from the group consisting of: an imager, a transmitter, a memory, a buffer and a circuit board.

11. The device according to claim 8, comprising connecting layers according to techniques selected from the group consisting of. Stacked tape carrier, Solder edge conductor bonding, Folded Flex Circuits, Thin Film Conductors, and stacked chips

12. A method of manufacturing an in vivo imaging device, the method comprising:
vertically stacking a plurality of components on a support, and
folding the support into an in vivo imaging device housing.

13. The method of claim 13, comprising inserting the folded circuit board into a swallowable capsule.

14. The method of claim 13, comprising interconnecting said components to each other.

15. The method of claim 13, comprising electrically interconnecting said components. 16. A method for vertically stacking an imager and a transmitter in an in-vivo imaging device comprising:
interconnecting the transmitter to a support by a conductive path; and
vertically interconnecting the imager to the transmitter.
